(19) United States
(12) Patent Application Publication
(10) Pub. No.: US 2005/0281885 A1
(43) Pub. Date: Dec. 22, 2005

(54) Method for treating inflammatory bowel disease by oral administration of IL-10

(76) Inventors: Nejat K. Egilmez, East Amherst, NY (US); Keith A. Sikora, Amherst, NY (US)

Correspondence Address: HODGSON RUSS LLP, ONE M & T PLAZA, SUITE 2000, BUFFALO, NY, 14203-2391, US

(21) Appl. No.: 10/873,068

(22) Filed: Jun. 21, 2004

(51) Int. Cl. A61K 38/20

(52) U.S. Cl. 424/490; 424/85.2

(57) ABSTRACT

The present invention provides compositions and methods for reducing the severity of inflammatory bowel disease. The composition comprises polymeric microspheres encapsulating IL-10. These polymeric microspheres can be administered orally to individuals to treat inflammatory bowel disease.

### FIELD OF THE INVENTION

The present invention relates generally to the field of inflammatory bowel disease and more particularly to a method of treating or reducing the severity of inflammatory bowel disease by oral administration of IL-10.

### DISCUSSION OF RELATED ART

Inflammatory bowel diseases (IBD), categorized as Crohn's disease (CD) and ulcerative colitis (UC), are chronic disorders of the gastrointestinal tract and are a significant cause of morbidity in Europe and the USA affecting 1 in 1000 people. Although the etiologies remain poorly understood, several genetic and environmental factors have been implicated in the pathogenesis of this disease (1,2). CD is characterized by transmural, patchy granulomatous inflammation of any part of the GI tract while UC is confined to the colon. The symptoms include diarrhea, abdominal pain, weight loss and nausea, and in extreme cases death can result from malnutrition, anemia and dehydration. The exact cause of IBD is not known however studies in humans and murine models of IBD suggest the involvement of abnormal T-cell responses to commensal microflora (3-5). The abnormal T-cell response in CD is typical of the TH1 type as assessed by elevated expression of IL-12, IFN-γ and TNF-α (4, 5). In UC the immune response is characterized by increased secretion of IL-5 but not IL-4 or IFN-γ (4, 5). In addition, increased expression of the inflammatory cytokines IL-1, IL-6, IL-8 and TNF-α is observed in the inflamed mucosa of IBD patients indicating abnormal macrophage and monocyte activity (5). This activity leads to amplification of the inflammatory cascade and secretion of more inflammatory mediators, destructive enzymes, and free radicals that cause tissue injury.

Agents currently used to treat IBD can be generally classified into two groups: compounds that suppress inflammation, and antibiotics that suppress the growth of gut flora. Non-steroidal anti-inflammatory agents such as 5-aminosalicylates appear to suppress inflammation by their inhibitory activity on cyclooxygenase and 5-lipoxygenase pathways as well as their ability to diminish antibody secretion and lymphocyte function (6, 7). Side effects include anorexia, dyspepsia, hemolysis neutropenia, pancreatitis nephritis (6). Corticosteroids are one of the longest used agents and are especially effective in active disease. They inhibit both adaptive and inflammatory immune responses. However, corticosteroid therapy has significant side effects some of which include adrenal suppression, glucose intolerance, hypertension, cataracts, infection, edema, impaired wound healing and osteoporosis (8). Other immune modulatory agents include thioguanine derivatives, methotrexate and cyclosporine. Again, side effects include most of the symptoms described above for 5-aminosalicylates and corticosteroids. Clinical trials have shown the efficacy of the antibiotic metronidazole in mild to moderate Crohn's disease (9, 10), in treatment of perianal disease (9) and in postsurgical prophylaxis (11). Ciproflaxin has been used to treat active disease in combination with Metronidazole (12). Common side effects include anorexia, nausea and peripheral neuropathy. While these agents can lead to temporary relief from the symptoms of disease, complete mucosal healing has not been observed. In fact about 30% of the patients are refractory to traditional treatments.

While the conventional therapies outlined above are effective in the treatment of active disease, disease remission is short-term and maintenance therapy is not effective over the long-term in the majority of the patients. More than 70% of the patients with Crohn's disease and 20-25% of the patients with ulcerative colitis undergo surgery within 20 years of diagnosis (1). Indications for surgery include intractable disease, toxic megacolon, massive hemorrhage, obstruction resulting from stricture and cancer. Thus, development of more effective novel therapies for long-term treatment and maintenance therapy is essential to improving the quality of life for IBD patients.

A third group of agents which have emerged recently involve biological response modifiers (BRM), i.e. biological macromolecules, that target the inflammatory lymphocytes and the cytokines produced by these cells in the GI tract of IBD patients. One such molecule, infliximab, a chimeric anti-human TNF α antibody, was approved for clinical use by the FDA in 1998 and has shown a high rate of response accompanied by significant mucosal healing in Crohn's disease (13). Treatment of patients with active disease that was refractory to conventional treatment also showed marked disease amelioration with long-term remissions (14). Other studies have shown significant healing of fistulas in Crohn's disease after infliximab therapy (15). The side effects observed have been favorable with a somewhat higher rate of upper respiratory tract infections and infrequent acute infusion reactions with short-term treatment (15). In some patients, re-treatment resulted in adverse reactions including rash, myalgia, fever, arthralgias and facial swelling due to the presence of high levels of anti-infliximab antibodies. Other emerging BRMs include the cytokine IL-10 which is a potent inhibitor of TH1 type responses as well as monocyte/macrophage activation (16), and the cytokine IL-11 which can inhibit macrophage effector function through the suppression of nuclear factor NFKB (17).

IL-10 is an 18-kilodalton cytokine produced by subsets of T- and B-cells, i.e macrophages and monocytes (18). IL-10 acts to suppress inflammation resulting from both antigen-specific and innate immune responses, by suppressing a)TH1 T-cell activity and the production of IL-12, IFN-γ and IL-2, b) by diminishing monocyte and macrophage activity and the production of IL-1, TNF-α and IL-6 and c) by reducing monocyte HLA class-II, CD80 and CD86 expression (18).

The importance of IL-10 in the mucosal immune regulation has been demonstrated by the development of IBD in mice lacking IL-10 (19). The IL-10 knock-out mice develop anemia, lose weight and die prematurely as a result of chronic enterocolitis (19-21). High levels of IFN-γ and TNF-α are detected in the intestinal explants of these mice indicating abnormal TH1 T-cell and monocyte/macrophage activity similar to that seen in human IBD patients. The histological alterations in the intestinal mucosa are also reminiscent of human IBD.

Treatment of IL-10 knock-out mice with antibodies against TNF-α, IFN-γ or IL-6 diminishes disease but does not cure it, even in young animals with minimal disease (21). Administration of recombinant IL-10 (i.v. or i.p.) in contrast to antibody treatment, shows a significant effect (21, 22). The mice gain weight, anemia is reduced and survival is enhanced. In young mice IL-10 can completely prevent the onset of disease (22).

Based on the encouraging results obtained in the murine studies, the safety and efficacy of IL-10 for the treatment of human IBD has been evaluated in several Phase I trials. In an initial open study, intravenous administration of recombinant human IL-10 for 7 consecutive days to CD patients resulted in disease remission in 50% of the patients as compared to 23% in the placebo group (23). In a second study involving patients with mild to moderate disease, multiple daily doses of IL-10 were administered subcutaneously for 28 days in the absence of any other treatment (24). The greatest effect was seen at a dose of 5 μg/kg with 29% remission compared to 0% in placebo. Higher doses were less effective and were associated with side-effects including headaches, fever, fall of hematocrit and thrombocytopenia. A third study duplicated the dosing regimen of the second but was performed in patients with chronic, active and steroid-resistant disease. Remission rates of 35% and 23% were observed in the treatment and placebo groups, respectively (25). Patients in the first and the third studies received other medication concurrently with IL-10 which accounts for the high rate of remissions seen in placebo groups.

While bolus delivery of soluble IL-10 improved disease status in the initial trials, the short in vivo half-life (1.5-3 hours) of IL-10 necessitated frequent administration of the cytokine. In addition, significant dose-dependent side-effects were observed. As a result, there is a continuing need for developing more efficient and safer modalities of use of IL-10 for IBD. Oral administration of soluble IL-10 has not heretofore been a viable option since labile proteins are quickly degraded in the highly acidic and protease-rich environment of the stomach. IL-10 formulations capable of surviving the stomach acids/degradative enzymes have not been described. Genetically-modified bacteria producing IL-10 have been tested in murine models with modest success (26). However the immunogenic nature of foreign bacteria and the safety concerns associated with administering genetically-modified bacteria into the body still remain. In one pre-clinical study, gelatin microspheres containing IL-10 were administered rectally which resulted in improvement of histological disease scores in a mouse model of IBD. However, these authors cautioned against the use of poly(DL-Lactic acid) microspheres for the delivery of protein-based drugs such as various cytokines, because “the biologic activity of the proteins might be lost due to protein-polymer formation” (27).

Thus, efficient oral delivery of cytokines has not heretofore been achieved. Recently, an article by Shen highlights some problems encountered with oral delivery of peptides and proteins and suggests more work needs to be done to obtain efficient delivery and absorption (28).

### SUMMARY OF THE INVENTION

The present invention is based on the unexpected finding that oral administration of encapsulated IL-10 can be used for reducing the severity of IBD. Accordingly, the present invention provides compositions comprising encapsulated IL-10. The formulations comprise polymeric microspheres encapsulating IL-10. In one embodiment, the polymers for preparing the microspheres include polyanhidrides. These include polylactic acid (PLA), polylactide-co-glycolide (PLGA), polycaprolactone (PCL) and poly(fumaric-co-sebacic anhydride) (p(FA:SA). Accordingly, in one embodiment of the invention, a method is provided for reducing the severity of IBD by oral administration of a drug composition comprising IL-10 encapsulated in polymeric microspheres.

### BRIEF DESCRIPTION OF THE DRAWINGS

FIG. 1. Changes in serum amyloid A (SAA) levels with age in normal controls (wild type) and in IL-10 knockout mice at 4, 7 and 10 weeks of age.

FIG. 2. Representative histological staining for mucosal thickening/crypt hyperplasia for colonic sections obtained from wild type (Panel A) or IL-10 knockout (Panels B-F). Panels B, C, D, E and F are representative section of the colon with scores of 0, 1, 2, 3, 4 and 4 respectively. Magnification=40×.

FIG. 3. Average histological scores for wild type and IL-10 knockout mice at different ages. Colonic samples from 3 mice in each group (4, 7 and 10 weeks old) were scored for wild-type and IL-10 knockout mice from FIG. 2. A score of 0 indicates normal histology and a score of 4 indicates severe disease.

FIG. 4. In vitro release profile of murine recombinant IL-10 from polylactic acid microspheres after 24, 48 and 72 hours.

FIGS. 5 Changes in the body weights of mice fed twice a week blank microspheres, or microspheres containing different amounts of IL-10. Average body weights of mice in each group were determined once a week throughout the experiment (n=5 per group) The percent total increase in average body weight at 18-19 days is shown.

FIG. 6. SAA levels in pre- and post-treatment mice. Mice were fed twice a week for 3 weeks starting at 5 weeks of age (n=5 mice/group). Bars=standard deviation. The difference between the blank microsphere and 1 μg IL-10 microsphere groups (both in IL-10^{-/-} mice) is statistically significant (p=0.001). Rx indicates treatment.

FIG. 7. Histological scoring of the colons. Colons (ascending, transverse and descending) from 3 mice in each group were processed and scored for disease severity as in Table 1. The difference between the blank microsphere control and the 1 μg IL-10 microsphere group was statistically significant (p=0.001).

### DETAILED DESCRIPTION OF THE INVENTION

The present invention provides compositions and methods for reducing the severity of IBD by oral administration of IL-10 in an encapsulated oral formulation. The formulation of the present invention comprises encapsulating the IL-10 in polymer microspheres. The formulation can be used for reducing the severity of IBD.

The term “effective amount” refers to an amount sufficient to ameliorate one or more symptoms of IBD in an individual. The present invention can be used in humans and primates, rats, cats, dogs and the like. The effective amount for a particular individual may vary depending upon a number of factors including the overall health of the individual, the weight, age and similar factors.

A “symptom” refers to any subjective evidence of the disease including evidence perceived by the individual such as diarrhea, abdominal pain, fever, and weight loss, or an objective criteria such as abdominal mass, dehydration glossitis, aphthous ulcer, anal fissure, perianal fissure, anemia, malabsorption and iron deficiency, serum amyloid A levels, or histological evaluation of biopsy sample.

Determination of the appropriate dosage is well within the purview of clinicians using standard parameters. The appropriate dose may be given as a single administration or may be divided into smaller doses.

The polymeric microspheres of the present invention comprise polymers including hydrophilic polymers such as those containing carboxylic groups, such as poly(acrylic acid). Rapidly bioerodible polymers such as poly(lactide-co-glycolide), polyanhydrides, and polyorthoesters having carboxylic groups exposed on the external surface as their smooth surface erodes, are particularly useful. Other representative synthetic polymers include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, celluloses including alkyl cellulose, hydroxyalkyl celluloses, cellulose ethers, cellulose esters, and nitrocelluloses, polymers of acrylic and methacrylic esters, poly(lactide-co-glycolide), polyanhydrides, polyorthoesters blends and copolymers thereof.

In general, the polymeric microspheres are slow-release biodegradable particles. The particles should have adequate uptake in the GI tract and be such that the release rate provides for sufficient release of the drug. In one embodiment, the polymeric biospheres are bioadhesive which is considered to increase the transit time of the particles in the GI tract. In one embodiment, thermoplastic polyanhidride polymers are used. These include polylactic acid (PLA), polylactide-co-glycolide (PLGA), polycaprolactone (PCL) and poly(fumaric-co-sebacic anhydride) (p(FA:SA).

In one embodiment described herein, the microspheres contain blends of two or more biodegradable polymers, preferably poly(hydroxy acids) of different molecular weight and/or monomer ratio. For example, different molecular weight polymers can be blended to form a composition that has linear release over a defined period of time, ranging from at least one day to several days. Thus, the release window can be varied by adjusting the molecular weight of the polymers used.

While not intending to be bound by any particular theory, it is considered that bioadhesive microspheres improve absorption by prolonging the intestinal passage time of the drug and extend pharmacokinetic half-life by the slow, sustained release of the drug. Administration of the drug via dispersed slow-release vehicles may also reduce adverse side effects.

The polymeric microspheres can be prepared by well known technologies (see Mathiowitz et al. Controlled Release 5, 13-22 (1987); Mathiowitz, et al., Reactive Polymers 6, 275-283 (1987); and Mathiowitz, et al., J. Appl. Polymer Sci. 35, 755-774 (1988), U.S. Pat. No. 6,235,313). The selection of the method depends on the polymer selection, the size, external morphology, and crystallinity that is desired, as described, for example, by Mathiowitz, et al., Scanning Microscopy 4, 329-340 (1990); Mathiowitz, et al., J. Appl. Polymer Sci. 45, 125-134 (1992); and Benita, et al., J. Pharmn. Sci. 73, 1721-1724 (1984). The method should be such that IL-10 is encapsulated without being inactivated. An example is a phase inversion (PIN) method.

In the PIN method, nano-seized microspheres are fabricated by the spontaneous phase inversion of dilute polymer solutions that are quickly dispersed into an excess of non-solvent for the polymer. This method differs from other methods of encapsulation in that no stirring or agitation of the non-solvent bath is required. Moreover there are no aqueous phases involved in the process which provides for high encapsulation efficiencies for hydrophillic molecules.

The microspheres of the present invention can be administered in suspension. Pharmaceutically acceptable carriers for oral administration are known and determined based on compatibility with the polymeric material. The dosage and administration of IL-10 are well within the purview of those skilled in the art. In general, IL-10 can be administered at a dose of 100-300 ng/kg per patient, which can be administered, for example for 5 days every 3 weeks for 3-4 cycles. IL-10 is currently used clinically and therefore the dosage and administration regimens are well known. Those skilled in the art will recognize that the dosage for the present invention would be typically less than what is used for the soluble form since the encapsulated drugs are being delivered locally and in a slow release form.

The polymeric microspheres of the present invention are useful for treating individuals afflicted with IBD, or individuals at risk of developing IBD, with an effective amount of encapsulated IL-10.

In one embodiment, the polymeric microspheres encapsulating IL-10 may be administered orally to individuals in combination with other therapeutic agents such as corticosteroids, sulphasalazines and derivatives thereof, cytotoxic drugs such as cyclosporin A, mercaptopurine and azathiopurine. In addition, the polymeric microspheres may also be administered to individuals who are considered to be at risk of developing IBD. The polymeric microspheres may also be administered with other therapeutic approaches such as immunotherapy. An example of an immunotherapeutic approach for Crohn's disease is administration of infliximab, a chimeric anti-human TNF a antibody.

The composition of the present invention can also be administered to individuals as risk of developing IBD. Such risk assessment may be based on several factors known to those skilled in the art including environmental factors, heredity, diet and the like.

The following examples will further describe the present invention. It should be noted that these examples are illustrative and are not intended to be restrictive in any way.

### EXAMPLE 1

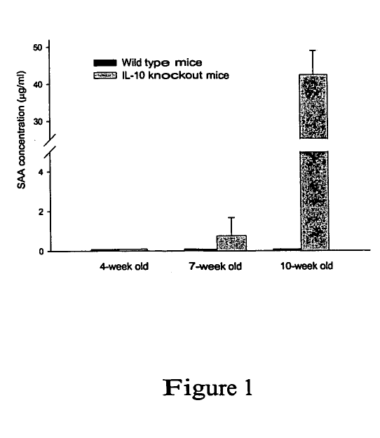

This example describes levels of serum amyloid A (SAA), an acute phase liver protein, as a marker for enterocolitis in normal control (wild type) and IL-10 knock-out mice. Mice were obtained at 4 weeks of age from Jackson Laboratories (Bar Harbor, Me.) and were maintained under standard conditions in the animal facility. Blood was collected from the mice at arrival (4 weeks of age), 7 and 10 weeks of age. Serum levels of SAA was determined with an SAA-specific ELISA (Biosource, Inc) for ages 4, 7 and 10 weeks. Some mice were sacrificed after bleeding at each time point for histological analysis of the intestines. FIG. 1 shows the changes in serum SAA levels with age in IL-10^{-/-} mice and normal controls. While not much difference is seen at 4 weeks of age, at 7 and 10 weeks of age the difference in SAA levels between control and IL-10 knock-out mice is quite evident.

### EXAMPLE 2

This example describes evaluation of another marker for Inflammatory Bowel Disease in IL-10 knock-out mice. Histological analysis of the colons obtained from the mice at different time points was performed to determine if SAA levels correlated with histological disease scores. For conducting histological analysis, animals were sacrificed, the colons (ascending, transverse and descending) removed, cut into 3-4 mm segments, fixed and embedded in paraffin. Sections (5 micron thick) were prepared, stained with hematoxylin and eosin and were analyzed under the microscope for histological scoring. Scoring was performed on paraffin sections obtained from segments throughout the colon (10-15 per mouse). Sections were scored at a magnification of 40×. Slides were scored relative to each other in a blinded fashion and the following criteria was developed:

A score of “0” indicates normal colonic architecture with distinct, non-inflamed villi, a lack of mononuclear cell infiltrates in lamina propria, a lack of epithelial hyperplasia and a lack of ulcerations.

A score of “1” indicates minimal thickening (up to 30% of normal) of bowel mucosa, 1-3 multi-focal mononuclear cell infiltrates per section in lamina propria, and 1-3 lumen ulcerations per section.

A score of “2” indicates moderate (between 30% to 50% of normal) thickening of bowel mucosa, multiple multi-focal (>3) mononuclear plus neutrophil cell infiltrates in lamina propria, multiple (>3) lumen ulcerations, 1-3 crypt hyperplasia per section.

A score of “3” indicates significant thickening (more than 50% of the normal) of bowel mucosa, large area of the mucosa involving mononuclear plus neutrophilic infiltrates, extensive ulceration invading submucosa, multiple (>3) crypt hyperplasia per section.

A score of “4” indicates complete loss of bowel mucosa architecture with mucosa filling the lumen, extensive mononuclear plus neutrophilic infiltrates throughout the section, transmural ulcerations with crypt hyperplasia and abscesses.

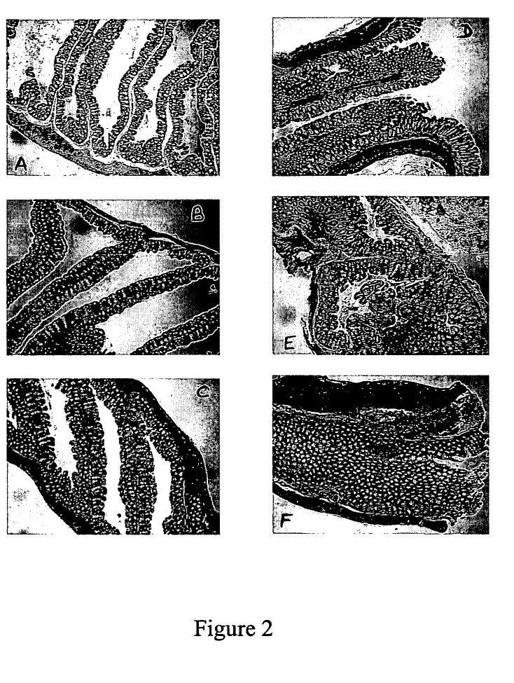

An illustration of sections assigned scores ranging from 0 to 4 is shown in Panels A-F of FIG. 2. A summary of the criteria is presented in Table 1.

TABLE 1
Criteria for Histological Scores.

Score

| Criteria | 0 | 1 | 2 | 3 | 4 |
|---|---|---|---|---|---|
| Bowel mucosa | Normal | Minimal | Mild- | Moderate- | Severe |
| thickening | (wt) | | moderate | severe | |
| Lymphocyte | Normal | Mild | Mild- | Moderate- | Severe |
| infiltration | (wt) | | moderate | severe | |
| Bowel lumen | None | Minimal | Mild | Moderate | Severe |
| ulcerations | (wt) | | | | |
| Crypt | None | None | Minimal | Mild | Moderate |
| hyperplasia | (wt) | | | | |
| Crypt | Absent | Absent | Absent | Absent | Present |
| abcesses | (wt) | | | | |
| Transmural | Absent | Absent | Absent | Absent | Present |
| inflammation | (wt) | | | | |

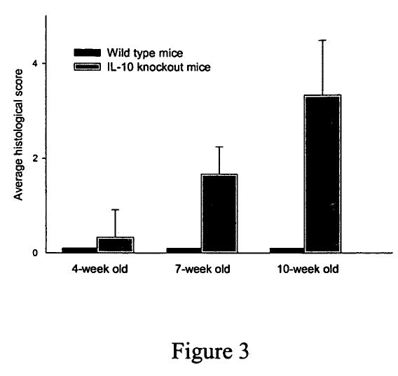

To determine if a correlation was present between the severity of the disease as measured by SAA levels and histological scores in the IL-10 knock-out mice, the following experiment was conducted. Three of ten mice (IL-10 knockout and wild type controls) were sacrificed upon arrival at 4 weeks (immediately after bleeding), 3 at 7 weeks of age and the rest at 10 weeks of age. Histological scores on the fixed and stained colonic sections were obtained as described above. The results are shown in FIG. 3. While not much difference is observed between the wild type and IL-10 knock-out mice at 4 weeks, the difference is quite pronounced at 7 and 10 weeks.

The results from SAA analysis and histological scoring demonstrate that the IL-10 knockout mice start developing disease sometime between 4-7 weeks of age and have established disease at 10 weeks of age. These data also establish that the SAA and histological scoring system correlate well thus providing two independent markers for monitoring disease development. Accordingly, in the following examples, the SAA levels and/or histological analysis was carried out to evaluate the effect of encapsulated IL-10 on IBD.

### EXAMPLE 3

This example describes the preparation of PLA microspheres comprising IL-10. A phase inversion nanoencapsulation technique was used for encapsulation of cytokines as follows. One milligram recombinant murine IL-10 (Peprotech, Inc.) in 0.2 ml phosphate buffered saline was mixed with 0.01 ml of bovine serum albumin solution (10% w/v in distilled water, Sigma Chemical Co., St. Louis, Mo.,), 0.0025 ml of Tween-20 (10% v/v in distilled water, Mallinckrodt, Paris, Kans.) and polylactic acid (PLA, 50 mg of MW 24,000 and 50 mg of MW 2,000 in 2 ml of methylene chloride, Birmingham Polymers, Inc, Birmingham, Ala.). The mixture was vortexed for 10 seconds and flash-frozen. The frozen emulsion was lyophilized for 48 hours, re-dissolved in methylene chloride and discharged into petroleum ether for production of microspheres. The microspheres were recovered by filtration through a 2.7 micron filter and lyophilized overnight for complete removal of solvent. The final formulation contained 1% BSA (wt/wt) and 0.5% murine IL-10 (w/w). Scanning electron micrographs demonstrated that the microspheres were 1-5 μm in diameter and were easily injectable with a 28.5 gauge needle.

### EXAMPLE 4

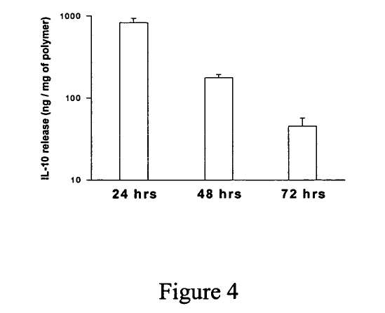

Subsequent to establishment of disease progression markers, the following experiment was performed to determine whether orally administered IL-10-encapsulated polylactic acid microspheres could be used to deliver IL-10. IL-10 microspheres prepared as described in Example 3, were first tested to determine whether the cytokine was released in a sustained fashion. Briefly, the microspheres were weighed out, hydrated and placed in culture medium in a 96-well culture plate and transferred to 37° C. CO₂ incubator. The culture medium was changed daily for 3 days and the amount of IL-10 in each supernatant sample was determined by ELISA (Biosource, Inc., Camarillo, Calif.). The results are shown in FIG. 4. The results demonstrate that IL-10 is released from the microspheres in a sustained fashion for at least 3 days.

### EXAMPLE 5

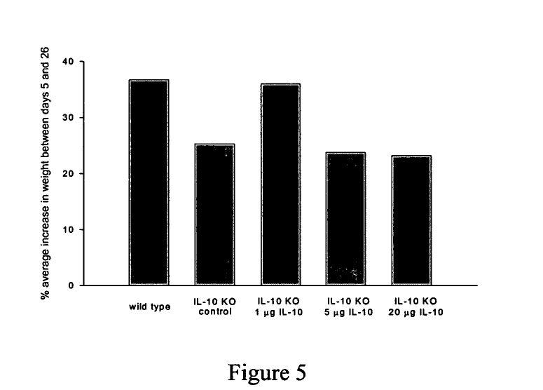

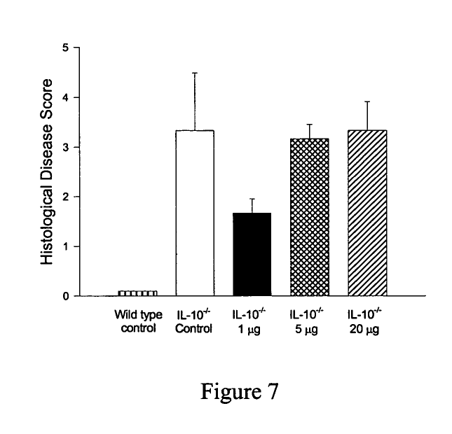

This example demonstrates the in vivo effect of encapsulated IL-10 in IL-10 knockout mice. Mice were obtained from Jackson Laboratories (Bar Harbor, Me.) at the age of 4 weeks and were maintained under standard conditions for one week. They were then fed twice a week for 3 weeks either with blank microspheres or with microspheres containing 1, 5 or 20 μg of IL-10, prepared as described in Example 3. An additional control group included wild type C57B1/6J mice that were fed blank microspheres. Serum samples were collected one day prior to the first feeding and 2 days after the last feeding. Mice were then sacrificed and the colons were processed for histological scoring. The changes in the body weights of mice in each group were also monitored during the experiment. The results are shown in FIGS. 5, 6 and 7.

FIG. 5 shows the percent total increase in average body weight at the end of the experiment for each group. The weight gain in the IL-10 knock-out control group was less than in the wild type (WT) group. A dose of 1 μg IL-10 was able to overcome the suppression of weight gain. These results indicate that administration of an effective amount of encapsulated IL-10 twice a week suppressed the overall weight gain pattern as compared to wild type controls.

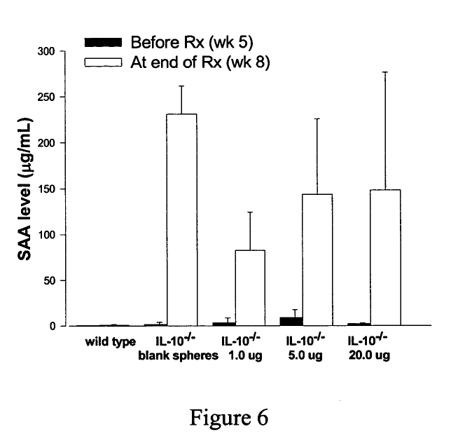

We next monitored the serum SAA levels in each group. Feedings of IL-10 were twice a week for 3 weeks starting at 5 weeks of age (n=5 mice/group). Bars=standard deviation. The results are shown in FIG. 6. The difference between the blank microsphere and 1 μg IL-10 microsphere groups (both in IL-10^{-/-} mice) is statistically significant (p=0.001).

The results shown in FIG. 6 establish that oral administration of IL-10 microspheres suppressed the development of enterocolitis in young IL-10 deficient mice as indicated by SAA levels. Interestingly, a biphasic response was observed in the effect of IL-10 both with respect to weight gain and SAA levels. Thus, the efficacy of treatment appeared to be dose-dependent in that, higher doses of IL-10 were not as effective in reducing SAA levels.

Histological analysis and scoring of the colons for severity of disease was then performed to confirm the SAA results. Colonic sections from mice in each group were evaluated in a blinded fashion and were scored for disease severity using the guidelines described in Example 2 and Table 1. Colons from 3 mice in each group were processed and scored for disease severity as described above (Table 1). The difference between the blank microsphere control and the 1 μg IL-10 microsphere group was highly significant (p=0.001).

The results from the histological analysis of colon samples confirm the patterns observed in FIG. 6 where treatment of mice with microspheres containing 1 μg of IL-10 was effective in suppressing disease development but this effect was reduced at higher doses as indicated by SAA levels. Thus the results shown in FIGS. 5, 6 and 7, where 3 independent disease markers are monitored, collectively demonstrate that oral feeding of mice with IL-10 microspheres can suppress disease development.

While this invention has been described through specific examples, minor modifications to the specific embodiments described here will be apparent to those skilled in the art and are intended to be within the scope of this invention.

### REFERENCES

1. Stotland et al., (2000). Med. Clinics of North America 84: 1107-1124.
2. Orchard et al., (2000). Scand. J. Immunol. 51: 10-17.
3. Strober et al., (1998). Ann. Intern. Med. 128: 848-856.
4. Macdonald et al., (2000). Scand. J. Immunol. 51: 2-9.
5. Papadakis et al., (2000). Annl. Rev. Med. 51: 289-298.
6. Sands (2000). Gastroenterology 118: S68-S82.
7. Grisham (1994). Lancet 344: 859-861.
8. Angeli et al., (1999). Ann. NY Acad. Sci. 876: 210-220.
9. Sutherland et al., (1991). Gut 32: 1071-1075.
10. Ursing et al., (1982). Gastroenterology 83: 550-562.
11. Jakobovits et al., (1984). Am. J. Gastroenterol. 79: 533-540.
12. Rutgeerts et al., (1995). Gastroenterology 108: 1617-1621.
13. Van Dullemen et al., (1995). Gastroenterology 109: 129-135.
14. Targan et al., (1997). N. Engl. J. Med. 337: 1029-1035.
15. Present et al., (1999). N. Engl. J. Med. 340: 1398-1405.
16. Narula et al., (1998). In: Therapeutic Strategies for Modulating the Inflammatory Diseases, pgs. 57-65, Birkhäuser Verlag, Basel/Switzerland.
17. Trepicchio et al., (1997). J. Immunol. 159: 5661.
18. Narula et al., (1995). In: R. Mertelsmann and F. Herrmann (eds): Hematopoietic Growth Factors in Clinical Applications, 2^{nd} Edition, Marcel Dekker Inc. pp 295-314.
19. Kühn et al., (1993). Cell 75: 263-274.
20. Rennick et al., (1995). Clin. Immunol. Immunopathol. 76: S174-S178.
21. Rennick et al., (1997). J. Leukocyte Biol. 61: 389-396.
22. Berg et al., (1996). J. Clin. Invest. 98: 1010-1020.
23. Van Deventer et al., (1997). Gastroenterology 113: 383-389.
24. Fedorak et al., (1998). Gastroenterology 114: A974.
25. Schreiber et al., (1998). Gastroenterology 114: A1080.
26. Steidler et al., (2000). Science 289:1352-1355.
27. Nakase et al. (2002) J. Pharmacol. Exp. Tharap. 301(1):59-65.
28. Shen (2003), DDT, vol. 8, no. 14, July 2003

1. A method of reducing the severity of inflammatory bowel disease comprising the step of orally administering to an individual in need of treatment, a formulation comprising polymeric microspheres encapsulating IL-10, wherein said oral administration of the encapsulated IL-10 is effective in ameliorating at least one symptom of the inflammatory bowel disease.

2. The method of claim 1, wherein the polymer is a polyanhydride.

3. The method of claim 1, wherein the polyanhydride is selected from the group consisting of polylactic acid, polylactide-co-glycolide, polycaprolactone and poly(fumaric-co-sebacic anhydride).

4. The method of claim 1, wherein the polymeric microspheres are prepared by the phase inversion method.

5. The method of claim 1, wherein the amount of IL-10 administered is about 100-300 ng/kg.

6. The method of claim 1, wherein the inflammatory bowel disease is Crohn's disease.

7. The method of claim 1, wherein the inflammatory bowel disease is ulcerative colitis.

8. The method of claim 1, wherein the polymer of the polymeric microspheres is polylactic acid.

9. The method of claim 1, wherein the polymer of the polymeric microspheres is poly(fumaric-co-sebacic anhydride).

10. The method of claim 1, wherein the polymeric microspheres are administered to the individual in combination with an agent selected from the group consisting of corticosteroids, sulphasalazines and derivatives thereof, and cytotoxic drugs.

11. The method of claim 1, wherein the polymeric microspheres are administered to the individual in combination with immunotherapy.

12. A method of reducing the levels of serum amyloid A or the histological score of a biopsied colonic section in an individual diagnosed with inflammatory bowel disease comprising the step of orally administering to an individual a formulation comprising polymeric microspheres encapsulating a drug composition comprising IL-10, wherein said oral administration of the formulation is effective in reducing said serum amyloid levels or the histological score of the biopsied colonic section.

13. The method of claim 12, wherein the polymer of the polymeric microspheres comprises a polyanhydride.

14. The method of claim 13, wherein the polyanhydride is selected from the group consisting of polylactic acid, polylactide-co-glycolide, polycaprolactone and poly(fumaric-co-sebacic anhydride).

15. The method of claim 12, wherein the polymeric microspheres are prepared by the phase inversion method.

16. The method of claim 12, wherein the amount of IL-10 administered is about 100-300 ng/kg.

17. The method of claim 12, wherein the inflammatory bowel disease is Cronh's disease.

18. The method of claim 12, wherein the inflammatory bowel disease is ulcerative colitis.

19. The method of claim 12, wherein the polymer in the polymeric microspheres is polylactic acid or poly(fumaric-co-sebacic acid) and the encapsulated agent is sulindac.

20. The method of claim 12, wherein the polymer in the polymeric microspheres is polylactic acid.

21. The method of claim 12, the polymeric microspheres are administered in combination with an agent selected from the group consisting of corticosteroids, sulphasalazines and derivatives thereof, and cytotoxic drugs.

22. The method of claim 12, wherein the polymeric microspheres are administered to the individual in combination with immunotherapy.
